(19) United States
(12) Patent Application Publication
(10) Pub. No.: US 2008/0269865 A1
(43) Pub. Date: Oct. 30, 2008

(54) Custom Length Stent Apparatus

(75) Inventors: David W. Snow, San Carlos, CA (US); Robert George, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: XTENT, INC., Menlo Park, CA (US)

(21) Appl. No.: 11/462,951

(22) Filed: Aug. 7, 2006

(51) Int. Cl. A61F 2/84

(52) U.S. Cl. 623/1.11

(57) ABSTRACT

Apparatus and methods for delivering prosthetic segments to a body lumen include an elongated flexible member having both proximal and distal ends along with a plurality of prosthetic segments releasably arranged axially along the elongated flexible member near the distal end. Additionally, the apparatus has a sheath slidably disposed over at least a portion of the prosthetic segments and an outer shaft slidably disposed over at least a portion of the sheath. A separator is disposed on the outer shaft and is biased into engagement with at least one prosthetic segment so that the outer shaft may be retracted to separate a proximal group of prosthetic segments from a distal group of prosthetic segments which are to be deployed into the body lumen. The sheath is positionable between the separator and the prosthetic segments to selectively disengage the separator from the prosthetic segments.

### BACKGROUND OF THE INVENTION

1. Field of the Invention

This invention relates generally to medical apparatus and methods, and more specifically to vascular catheters, stents and stent delivery systems for use in the coronary arteries and other vessels.

Stenting is an important treatment option for patients with vascular occlusive disease. The stenting procedure involves placing a tubular prosthesis at the site of a lesion, typically within a diseased coronary artery. The procedure is performed in order to maintain the patency of the artery and is often performed after a primary treatment such as angioplasty. Early stent results suffered from high rates of restenosis, i.e. the tendency for the stented coronary artery to become re-occluded following implantation of the stent. However, in recent years, restenosis rates have decreased substantially, due in part to drug eluting stents as well as other improvements in stent delivery methods and stent technology. As a result, the number of stent related procedures being performed worldwide continues to dramatically increase.

Stents are typically either self-expanding or balloon expandable and they are delivered to the coronary arteries using long, flexible vascular catheters typically inserted percutaneously through the patient's femoral artery. For self-expanding stents, the stent is simply released from the delivery catheter and it resiliently expands into engagement with the vessel wall. For balloon expandable stents, a balloon on the delivery catheter is expanded which expands and deforms the stent to the desired diameter, whereupon the balloon is deflated and removed, leaving the stent in place.

Current stent delivery technology suffers from a number of drawbacks which can make delivery of stents challenging. In particular, current stent delivery catheters often employ stents having fixed lengths. The proper selection of fixed length stents requires accurate knowledge of the lesion length being treated. While lesion length may be measured prior to stent deployment using angiography and fluoroscopy, these measurements are often inaccurate. Thus, if an improperly sized stent is introduced to a treatment site, the delivery catheter and stent must be removed from the patient and replaced with a different device having the correct size. This prolongs the procedure, increases waste and results in a more costly procedure.

The use of “custom length” stents as an alternative to fixed length stents has been proposed. One such approach for providing a custom length stent has been to use segmented stents for treatment in which only some of the stents are deployed for treatment. Several exemplary systems are described in several copending, commonly assigned applications which are listed below. In these systems, the stent segments are deployed by selective advancement over the delivery catheter. After delivering an initial group of segments, the catheter may be repositioned to a new treatment site and a further group of segments can then be deployed. These systems can enable treatment of multiple lesions with a single device and may contain up to fifty segments. While this technology represents a significant improvement over earlier stent delivery systems, in the case of smaller, more focal lesions or single lesions, only a small number of stent segments are needed and thus there is considerable waste when a large number of stent segments remain undeployed and end up being discarded at the end of the procedure.

Another challenge with existing “custom length” stent delivery systems is that to deliver multiple stent segments to multiple lesion sites requires an intricate delivery system that can be somewhat complex to use. Thus, a simpler delivery system that allows length customization with fewer prosthetic segments on the delivery catheter is desirable, especially for use in treating a single lesion. It is also desirable to protect stent segments on the delivery system from being improperly displaced, deformed or damaged during delivery and deployment.

For the above reasons as well as others, it would be desirable to provide improved prosthetic stents and delivery catheters. It would be particularly desirable to provide catheters which enable stent length to be customized yet have a minimal quantity of stent segments so as to treat common lesion lengths while minimizing stent segment waste. It is also desirable to provide a delivery system that is flexible and can track torturous vessels and that has a simple construction and is less costly and easy to use in deploying a selectable number of stent segments to a single treatment site.

2. Description of the Background Art

Prior publications describing catheters for delivering multiple segmented stents include: U.S. Publication Nos. 2004/0098081, 2005/0149159, 2004/0093061, 2005/0010276, 2005/0038505, 2004/0186551 and 2003/013266. Prior related unpublished co-pending U.S. patent applications include Ser. No. 11/148,713, filed Jun. 8, 2005 (Attorney Docket No. 14592.4002), entitled “Devices and Methods for Operating and Controlling Interventional Apparatus”; Ser. No. 11/148,545, filed Jun. 8, 2005 (Attorney Docket No. 14592.4005), entitled “Apparatus and Methods for Deployment of Multiple Custom-Length Prosthesis”; Ser. No. 11/344,464, filed Jan. 30, 2006 (Attorney Docket No. 021629-003500US), entitled “Apparatus and Methods for Deployment of Custom-Length Prostheses”; Ser. No. 60/784,309, filed Mar. 20, 2006 (Attorney Docket No. 021629-003600US), entitled “Apparatus and Methods for Deployment of Linked Prosthetic Segments”; Ser. No. ______, filed (Attorney Docket No. 021629-003800US), entitled “Custom Length Stent Apparatus”; and Ser. No. ______, filed (Attorney Docket No. 021629-004000US), entitled “Custom Length Stent Apparatus.” The full disclosures of each of these patents and applications are incorporated herein by reference.

### BRIEF SUMMARY OF THE INVENTION

The invention generally provides for the delivery of prosthetic segments with a flexible delivery catheter capable of navigating torturous vessels such as the coronary arteries. The delivery catheter permits deployment of a selectable number of prosthetic segments at a single treatment site, thus allowing customization of prosthesis length while the delivery catheter is in a body lumen at a treatment site. Customization of prosthesis length in situ permits better matching of the prosthesis length to the lesion length being treated. The delivery catheter has a simplified design including a control mechanism on the catheter handle for selecting prosthetic segments for deployment and a stent valve or separator on the distal end of an outer shaft that facilitates deployment of the selected group of stent segments. A sheath protects the prosthetic segments from damage during delivery and deployment.

The terms “stent” and “stenting” are defined to include any of the array of expandable prostheses and scaffolds which are introduced into a lumen at a target treatment site and expanded in situ thereby exerting a radially outward force against the lumen wall. The prosthesis of the present invention comprises a closed or an open lattice structure and is typically fabricated from a malleable or elastic material. When a malleable material is used, such as stainless steel, gold, platinum, titanium, cobalt chromium and other alloys, the stent segments are typically expanded by balloon inflation, causing plastic deformation of the lattice so that it remains permanently deformed in the open position after deployment. When formed from an elastic material, including superelastic materials such as nickel-titanium alloys, the lattice structures are commonly constrained radially during delivery and upon deployment the constraining structure is removed, allowing the prosthesis to “self-expand” at the target site. The terms “stent,” “prosthetic segment” and “stent segments” refer broadly to all radially expansible stents, grafts, and other scaffold-like structures which are intended for deployment within a body lumen.

In a first aspect of the invention, an apparatus for delivering prosthetic segments in a body lumen comprises an elongated flexible member with proximal and distal ends and a plurality of prosthetic segments releasably arranged axially along the elongated flexible member near the distal end. The apparatus also has a sheath that is slidably disposed over at least a portion of the prosthetic segments and an outer shaft that is slidably disposed over at least a portion of the sheath. A separator is disposed on the outer shaft and is biased into engagement with at least one prosthetic segment so that the outer shaft may be retracted to separate a proximal group of prosthetic segments from a distal group of prosthetic segments which are to be deployed in the body lumen. The sheath is also positionable between the separator and the prosthetic segments to selectively disengage the separator from the prosthetic segments.

In preferred embodiments, the separator is adapted to apply substantially greater axial force against the prosthetic segments when the separator is retracted proximally than when the separator is advanced distally. Often, the sheath is configured to engage the outer shaft such that retraction of the sheath retracts the outer shaft. Both the sheath and outer shaft typically comprise an annular flange that allows the two members to engage one another. The apparatus also usually includes an expandable member near the distal end of the elongate flexible member and typically the expandable member is a balloon.

Often, the prosthetic segments are balloon expandable, although they may also be self-expanding. Additionally, the prosthetic segments may carry a therapeutic agent such as an anti-restenosis drug which may be released from the segments. The segments are often in the range of size from about 2 mm to about 10 mm, although they typically have a length about 3 mm to about 6 mm. In some embodiments, the prosthetic segments may have two or more lengths, while in other embodiments, the segments are substantially the same length. Often, the prosthetic segments have interleaved ends in engagement with each other prior to deployment although the segments may also be spaced apart prior to deployment. Spacing the segments apart allows the separator to engage the prosthetic segments at their distal ends. The separator often comprises a plurality of resilient fingers projecting radially inward. At least some of these fingers may be inclined so that the free ends of the fingers point proximally allowing the fingers to pass over the prosthetic segments as the separator is advanced distally but to engage a prosthetic segment when the separator is retracted proximally. In some embodiments, some of the fingers are composed of metal, while they may also be composed of a polymer. Some of the fingers may comprise a radiused end that substantially matches the curvature of the surface of the prosthetic segments thereby providing greater contact surface so as to facilitate engagement between the prosthetic segments and the separator as the separator is retracted proximally while allowing the separator to pass over the prosthetic segments during distal advancement of the separator.

In other embodiments, the separator further comprises a hinge coupled to the fingers to allow the fingers to deflect radially and this facilitates passage of the separator over the sheath when the separator is advanced distally. Often the separator comprises an annular flange and this flange may be tapered or it can be a tapered conical nose. Other embodiments of the separator comprise a plurality of inclined ramps disposed on an inner surface of the outer shaft and these ramps may be separated by about ninety degrees. In still other embodiments, the separator may comprise a compliant sharp edge.

In another aspect of the present invention, a method for delivering prosthetic segments to a body lumen comprises introducing a plurality of prosthetic segments that are releasably arranged axially along an elongated flexible member, into a body lumen having a lesion with a lesion length at a first treatment site. An outer shaft having a separator is distally advanced relative to a group of prosthetic segments selected for delivery and the selected group typically has a combined length that matches the lesion length. A sheath, disposed between the separator and the prosthetic segments is then positioned until the separator is allowed to engage the prosthetic segments. The outer shaft is then retracted thereby creating a spacing between prosthetic segments in the selected group and one or more prosthetic segments remaining with the elongated flexible member. The selected group of prosthetic segments is then deployed at the first treatment site.

In still another aspect of the present invention, a method for selectively delivering prosthetic segments to a lesion in a treatment region in a body lumen comprises advancing a delivery catheter through the body lumen, which may be a blood vessel, to the treatment region. A plurality of prosthetic segments is often disposed axially along the delivery catheter. An outer shaft having a separator is distally advanced. A sheath disposed between the separator and the prosthetic segments is positioned until the separator is allowed to engage the prosthetic segments. The outer shaft can then be retracted proximally so as to create a spacing between prosthetic segments in the selected group and one or more prosthetic segments remaining with the elongate flexible member. Inflating a balloon disposed on the delivery catheter deploys the group of prosthetic segments while any remaining prosthetic segments stay with the delivery catheter.

Often, the sheath is configured to engage the outer shaft such that retraction of the sheath also retracts the outer shaft. Additionally, the prosthetic segments are typically balloon expandable, but they may also be self-expanding, and they are usually introduced into a blood vessel. Often, deploying the selected group of prosthetic segments comprises plastically deforming the selected group of prosthetic segments, and often this is accomplished with a balloon. Often, the plurality of prosthetic segments carries a therapeutic agent that is adapted to being released from the segments, and typically this agent is an anti-restenosis drug.

In some embodiments, the prosthetic segments have a length in the range from about 2 mm to about 10 mm, while in others, the segments have a length about 3 mm to about 6 mm. Often prosthetic segments have interleaved ends in engagement with each other prior to deployment, although the segments may also be spaced apart prior to deployment to allow the separator to engage the prosthetic segments at their distal ends.

Typically, the separator exerts substantially greater axial force against the prosthetic segments when the separator is retracted proximally than when the separator is advanced distally. In some embodiments, the separator comprises a plurality of resilient fingers projecting radially inward. Often, at least some of the fingers are inclined so that free ends of the fingers point proximally allowing the fingers to pass over the prosthetic segments as the separator is advanced distally but to engage a prosthetic segment when the separator is retracted proximally. Some of the fingers may be composed of metal, while in other embodiments, some of the fingers may be composed of a polymer.

In other embodiments, at least some of the fingers comprise a radiused end that substantially matches the curvature of the prosthetic segments thereby providing greater contact surface so as to facilitate engagement between the prosthetic segment and the separator as the separator is retracted proximally while allowing the separator to pass over the prosthetic segments during distal advancement of the separator. Some embodiments may include a separator that comprises a hinge coupled to the fingers. The hinge helps the fingers to deflect radially outward over the sheath when the separator is advanced distally. Often, the separator comprises an annular flange and the flange may be tapered or may have a tapered conical nose.

In still other embodiments, the separator comprises a plurality of inclined ramps that are disposed on an inner surface of the outer shaft. Often these ramps are separated by about ninety degrees. In yet other embodiments, the separator may comprise a sharp compliant edge.

These an other embodiments are described in further details in the following description related to the appended drawing figures.

### BRIEF DESCRIPTION OF THE DRAWINGS

FIG. 1 is a perspective view of a stent delivery system in accordance with one embodiment of the present invention.

FIGS. 2A-2F show selection and deployment of prosthetic stent segments in accordance with an exemplary embodiment.

### DETAILED DESCRIPTION OF THE INVENTION

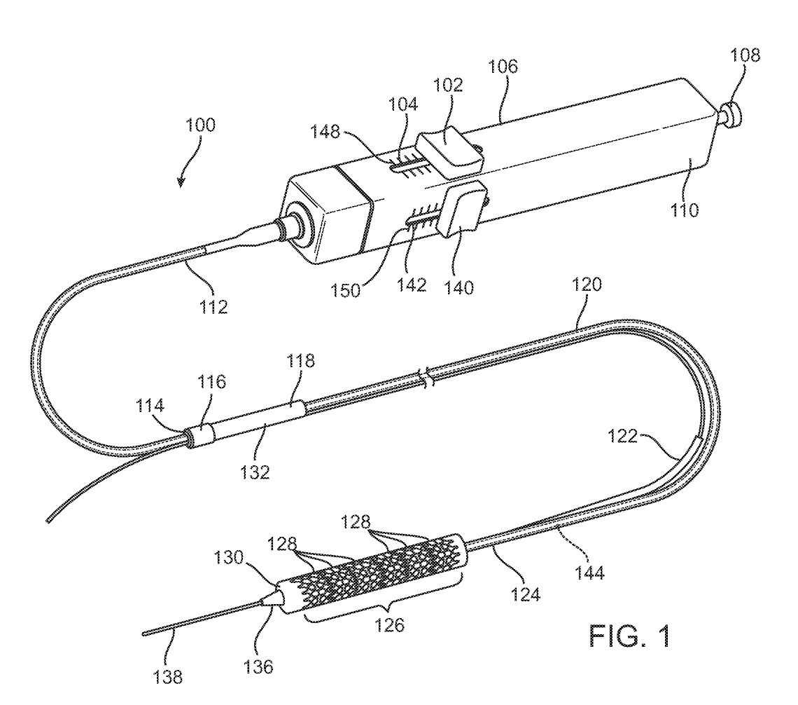

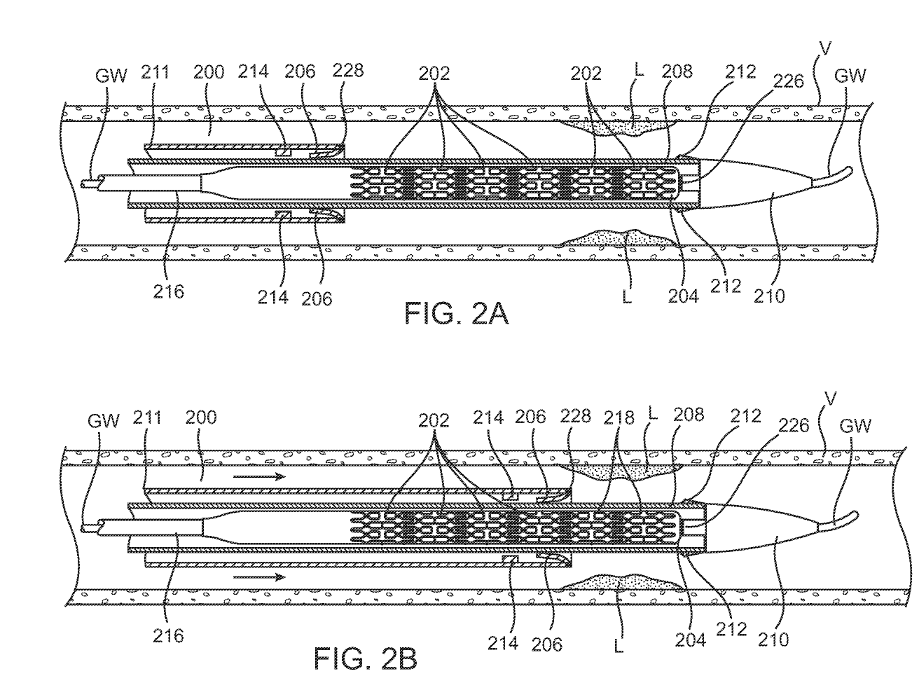

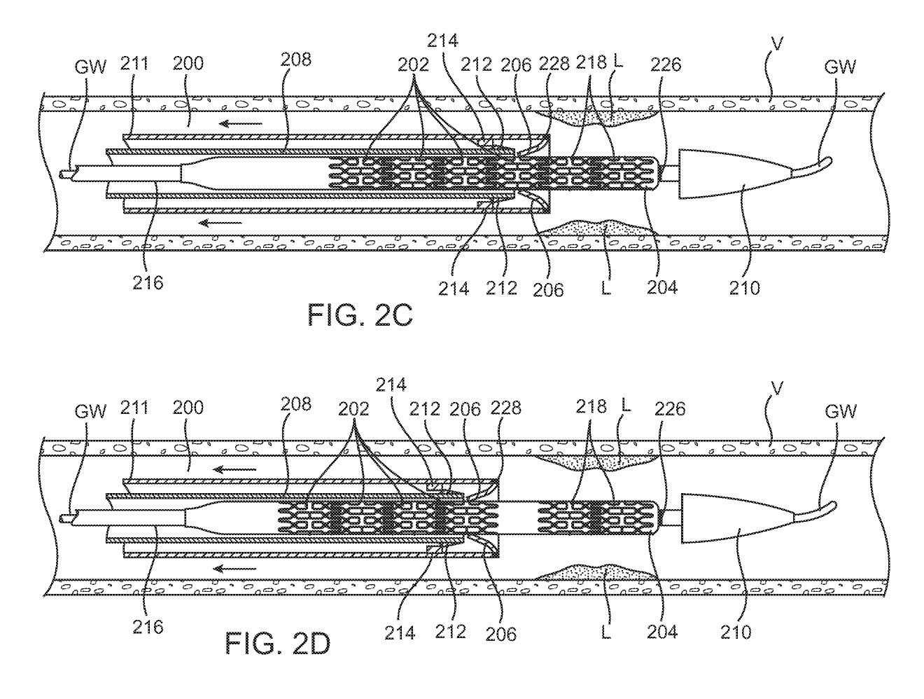

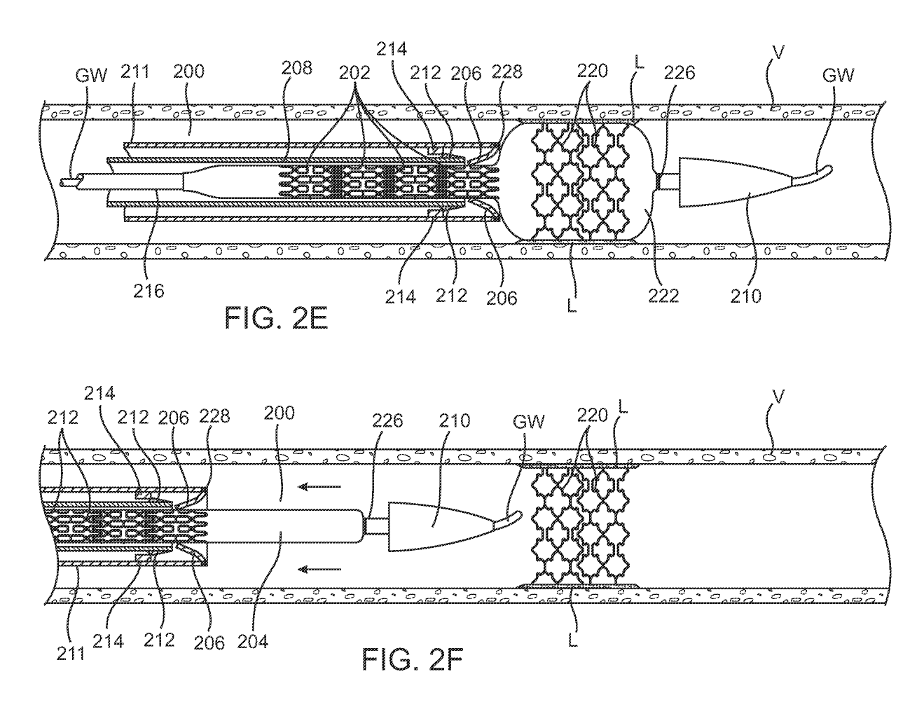

Referring now to FIG. 1, a stent delivery catheter 100 comprises a catheter shaft 120 with an outer shaft 124 slidably disposed over a sheath 144 which is in turn slidably disposed over an inner shaft 216 (seen in FIG. 2A). An inflatable balloon 130, is mounted on the inner shaft 216 and is exposed by retracting outer shaft 124 and sheath 144 relative to the inner shaft 216. A tapered nosecone 136, composed of a soft elastomeric material to minimize trauma to the vessel during advancement of the delivery catheter 100, is attached distally of the inflatable balloon 130. Prosthesis 126 comprises a plurality of prosthetic segments 128 mounted over the inflatable balloon 130 for expansion. Sheath 144 covers the prosthetic segments 128 and protects them from being damaged during delivery. A guidewire tube 122 is slidably positioned through shaft 124 and sheath 144 proximal to the inflatable balloon 130. A guidewire 138 is positioned slidably through guidewire tube 122, inflatable balloon 130 and nosecone 136, and extends distally thereof. FIG. 1 illustrates the stent delivery catheter 100 and FIG. 2A shows various elements of the delivery catheter 100 in greater detail.

In FIG. 2A, a stent delivery catheter 200 is slidably disposed over the guidewire GW into the vessel V so that the nosecone 210 is distal to the lesion L. The delivery catheter 200 has an expandable member 204 disposed over an inner catheter shaft 216 and stent segments 202 having interleaved ends in engagement with each other are disposed over the expandable member 204, which is a balloon in this embodiment. The stent segments 202 are covered by sheath 208. The sheath 208 protects stent segments 202 during delivery and also prevents unintended axial displacement of the segments 202. In this embodiment, six stent segments 202 are disposed on the stent delivery catheter 200. The prosthetic segments 202 are disposed over a balloon 204 near the distal end of the stent delivery catheter 200. A stent valve 206 is disposed on the inner surface of outer shaft 211 and is adapted to engage prosthetic segments 202 and facilitates their deployment. Additionally, sheath 208 has an annular flange or tab 212 disposed on its outer surface that is adapted to pass through the stent valve 206 during retraction or advancement and to engage the annular flange or tab 214 disposed on the inner surface of outer shaft 211

In FIG. 1, a handle 106 is attached to a proximal end 112 of the outer shaft 124. The handle performs several functions, including retracting and advancing the outer shaft 124 and sheath 144 thereby exposing prosthetic segments 128 and allowing the prosthetic segments 128 to be delivered. Additionally, using the handle 106 to displace the sheath 144 permits creation of a spacing between prosthetic segments 128 selected for delivery and the segments 128 that will remain with the delivery catheter 100. This gap or spacing between segments permits proper balloon inflation and will be described below in further detail along with the handle structure and operation.

Handle 106 includes a housing 110 which encloses the internal components of the handle 106. Handle 106 allows a physician operator to advance or retract outer shaft 124 and sheath 144, which determines the length of the prosthesis (number of segments) to be deployed. The handle 106 also permits connection of balloon 130 to an inflation source. The inner shaft 216 is preferably fixed to the handle housing 110, while both outer shaft 124 and sheath 144 are coupled to slide mechanisms 102 and 140, respectively. Slide mechanisms 102 and 140 allow both the outer shaft 124 and sheath 144 to be independently retracted and advanced relative to handle 106. An adaptor 108 is attached to handle 106 at its proximal end and is fluidly coupled to the inner shaft 216 in the interior of the housing of handle 106. The adaptor 108, preferably a Luer connector, is configured to be fluidly coupled with an inflation device which may be any commercially available balloon inflation device such as those sold under the trade name “Indeflator™,” manufactured by Abbott (formerly Guidant Corporation of Santa Clara, Calif.). The adaptor is in fluid communication with the inflatable balloon 130 via an inflation lumen in the inner shaft 216 to permit inflation of the inflatable balloon 130.

Additionally, a control mechanism on the handle 106 includes a slide mechanism 102 that translates along calibrated slot 104. Slide mechanism 102 is coupled with outer shaft 124 and is adapted to retract or advance the shaft 124 a selected distance. Initially, the distance is selected by advancing slide mechanism 102 distally along slot 104. This allows the physician operator to select the number of prosthetic segments 128 on the distal end of delivery catheter 100 that will be delivered. The slide mechanism 102 includes visual markers 148 so that an operator can easily determine how many stent segments have been selected. In preferred embodiments, slide mechanism 102 may have detents or a ratchet that provides audible or tactile feedback to the operator to facilitate operation of the stent delivery catheter 100 without requiring direct visualization during operation.

Handle 106 also comprises a second control mechanism 140 that translates along calibrated slot 142. Slide mechanism 140 is coupled with the sheath 144 and is adapted to retract or advance the sheath 144 a selected distance. After the number of prosthetic segments 128 has been selected as described above, slide mechanism 140 is retracted so as to cause proximal retraction of sheath 144. As sheath 144 is retracted, tabs 212 (FIG. 2A) engage with tabs 214 (FIG. 2A) on outer shaft 124. Further retraction of sheath 144 also retracts outer shaft 124 and exposes the prosthetic segments 128 selected for delivery and creates a spacing between the segments selected for delivery and the segments remaining with the delivery catheter 100. Slide mechanism 140 also includes visual markers 150 that help the physician with operation of the control mechanism 140. Additionally, the slide mechanism 140 may comprise detents or a ratchet that further assists physician operation by providing audible or tactile feedback. This series of steps is illustrated in FIGS. 2A-2F and is described in more detail below.

Additional details on materials and construction of other related handles are described in co-pending U.S. patent application Ser. No. 11/148,713, filed Jun. 8, 2005, (Attorney Docket No. 14592.4002), entitled “Devices and Methods for Operating and Controlling Interventional Apparatus,” and co-pending United States Publication No. 2005/0149159, entitled “Devices and Methods for Controlling and Indicating the Length of an Interventional Element,” and application Ser. No. ______, filed ______, 2006, (Attorney Docket No. 021629-003800US), entitled “Custom Length Stent Apparatus,” the full disclosures of which are incorporated herein by reference.

Both sheath 144 and outer shaft 124 along with guidewire 138 each extend through a slider assembly 132 slidably disposed on the catheter body 120 at a point between its handle 106 and expandable member 130. The slider assembly 132 is adapted for insertion into and sealing with a hemostasis valve, such as on an introducer sheath or guiding catheter, while still allowing relative movement of the outer shaft 124 and sheath 144 relative to the slider assembly 132. The slider assembly 132 includes a slider tube 118, a slider body 116, and a slider cap 114.

Outer shaft 124 and sheath 144 may be composed of any of a variety of biocompatible materials, such as but not limited to a polymer like PTFE, FEP, polyimide, Nylon or Pebax, and may be reinforced with a metallic or polymeric braid to resist radial expansion of inflatable balloon 130, and/or the like. Inflatable balloon 130 may be formed of a semi-compliant polymer such as Pebax, Nylon, polyurethane, polypropylene, PTFE or other suitable polymers. Additional aspects of the luminal prosthesis delivery system are described in U.S. patent application Ser. No. 10/306,813, filed Nov. 27, 2002 (Attorney Docket No. 021629-000320US); U.S. patent application Ser. No. 10/637,713, filed Aug. 8, 2003 (Attorney Docket No. 021629-000340US); U.S. patent application Ser. No. 10/738,666, filed Dec. 16, 2003 (Attorney Docket No. 021629-000510US); U.S. patent application Ser. No. 11/104,305, filed Apr. 11, 2005 (Attorney Docket No. 021629-003300US); and U.S. application Ser. No. 11/148,585, filed Jun. 8, 2005, the full disclosures of which are hereby incorporated by reference.

Delivery catheter 100 also includes a separator or “stent valve” disposed near the distal end of outer shaft 211 and an exemplary embodiment of this is seen in FIG. 2A. In FIG. 2A, outer shaft 211 is retracted so that its distal tip is proximal to the proximal-most end of the prosthetic stent segments 202. At the same time, sheath 208 is advanced fully distally, covering the plurality of prosthetic segments 202 which are disposed over expandable member 204. Expandable member 204 acts as a carrier which supports the prosthetic segments 202.

Separator 206 contacts and engages prosthetic segments 202. As shown in FIG. 2A, separator 206 includes proximally inclined resilient fingers configured to frictionally engage stent segments 202 when sheath 208 has been retracted and prosthetic segments 202 are exposed. Thus, when outer shaft 211 is retracted proximally, the separator 206 engages the prosthetic segments 202, while the separator 206 slides over the prosthetic segments 202 when the segments 202 are covered by sheath 208. The separator 206 may be a polymeric or metallic material integrally formed with outer shaft 211, or it may be bonded or otherwise mounted to the interior of the outer shaft 211. The geometry of separator 206 can also be toroidal with a circular or ovoid cross-section (like an O-ring) or the separator 206 may have another cross-sectional shape such as triangular, trapezoidal, pyramidal, or other shapes as described in embodiments discussed more fully herein below. The separator 206 can be a polymer such as silicone or urethane, sufficiently soft, compliant and resilient to provide frictional engagement with stent segments 202, in some embodiments without damaging any coating deposited thereon, including therapeutic drug coatings. The separator 206 extends radially inwardly a sufficient distance to engage the exterior of stent segments 202 with sufficient force to allow the stent segments not selected for delivery to be retracted proximally with outer shaft 211 so as to create a spacing relative to those stent segments selected for delivery. Other exemplary embodiments of separators along with additional aspects of separator 206 are described in U.S. patent application Ser. No. 10/412,714, filed Apr. 10, 2003 (Attorney Docket No. 021629-000330US); U.S. patent application Ser. No. 11/344,464, filed Jan. 30, 2006 (Attorney Docket No. 021629-003500US); and U.S. patent application Ser. No. ______, filed ______ (Attorney Docket No. 021629-004000US), the entire contents of which are incorporated herein by reference.

Prosthesis 126 is composed of one or more prosthetic segments 128. Prosthetic stent segments 128 are disposed over an inflation balloon 130. Each stent segment is about 2-20 mm in length, more typically about 2-10 mm in length and preferably being about 2-8 mm in length. Usually 2-20, more typically 2-10 and preferably 2-6 stent segments 128 may be positioned axially over the inflation balloon 130 and the inflation balloon 130 has a length suitable to accommodate the number of stent segments. Stent segments 128 may be positioned in direct contact with an adjacent stent segment so that segment ends are interleaved or there may be a spacing between segment ends. When the segments are spaced apart from one another, the spacing is typically between 0.5 mm and 1 mm. Furthermore, the stent segments 128 may be deployed individually or in groups of two or more at a single treatment site within the vessel lumen.

Prosthetic stent segments 128 are preferably composed of a malleable metal so they may be plastically deformed by inflation balloon 130 as they are radially expanded to a desired diameter in the vessel at the target treatment site. The stent segments 128 may also be composed of an elastic or superelastic shape memory alloy such as Nitinol so that the stent segments 128 self-expand upon release into a vessel by retraction of the sheath 124. In this case, an inflation balloon 130 is not required but may still be used for predilation of a lesion or augmenting expansion of the self-expanding stent segments (e.g. postdilation or tacking). Other materials such as biocompatible polymers may be used to fabricate prosthetic stent segments and these materials may further include bioabsorbable or bioerodable properties.

Stent segments 128 may have any of a variety of common constructions, such as but not limited to those described in U.S. patent application Ser. No. 10/738,666 filed Dec. 16, 2003 (Attorney Docket No. 02169-000510US), which was previously incorporated by reference. Constructions may include for example, closed cell constructions including expansible ovals, ellipses, box structures, expandable diamond structures, etc. In addition, the closed cells may have complex slotted geometries such as H-shaped slots, I-shaped slots, J-shaped slots, etc. Suitable open cell structures include zigzag structures, serpentine structures, and the like. Such conventional stent structures are well described in the patent and medical literature. Specific examples of suitable stent structures are described in the following U.S. patents, the full disclosures of which are incorporated herein by reference: U.S. Pat. Nos. 6,315,794; 5,980,552; 5,836,964; 5,527,354; 5,421,955; 4,886,062; and 4,776,337.

In preferred embodiments, prosthetic stent segments 128 may be coated, impregnated, infused or otherwise coupled with one or more drugs that inhibit restenosis, such as Rapamycin, Everolimus, Paclitaxel, analogs, prodrugs, or derivatives of the aforementioned, or other suitable agents, preferably carried in a durable or bioerodable polymeric carrier. Alternatively, stent segments 128 may be coated with other types of drugs or therapeutic materials such as antibiotics, thrombolytics, anti-thrombotics, anti-inflammatories, cytotoxic agents, anti-proliferative agents, vasodilators, gene therapy agents, radioactive agents, immunosuppressants, chemotherapeutics and/or stem cells. Such materials may be coated over all or a portion of the surface of stent segments 128, or stent segments 128 may have a porous structure or include apertures, holes, channels, or other features in which such materials may be deposited.

Referring now to FIGS. 2A-2F, the deployment of selected prosthetic segments to treat a stenotic lesion is shown in accordance with an exemplary embodiment. While the embodiment will be described in the context of a coronary artery stent procedure, it should be understood that the invention may be employed in any variety of blood vessels and other body lumens in which stents or tubular prostheses are deployed, including the carotid, femoral, iliac and other arteries and vein, as well as non-vascular body lumens, such as the ureter, urethra, fallopian tubes, the hepatic duct and the like. A guide catheter (not illustrated) is first inserted into a peripheral artery such as the femoral artery, typically using a percutaneous procedure such as the Seldinger technique or by surgical cutdown, and then advanced to the ostium of the right or left coronary artery. Guidewire GW is then inserted through the guiding catheter and advanced to the target vessel V, where the lesion L to be treated is located. The proximal end of guidewire GW is then inserted through nose cone 210, through the catheter shaft 216 and exits guidewire tube 122 (seen in FIG. 1) which is outside the patient's body.

FIG. 2A shows stent delivery catheter 200 slidably advanced over the guidewire GW into the vessel V so that the nosecone 210 is distal to the lesion L. The delivery catheter 200 has an expandable member 204 disposed over a catheter shaft 216 and stent segments 202 having interleaved ends in engagement with each other are disposed over the expandable member 204, which is a balloon in this embodiment. The stent segments 202 are covered by sheath 208. The sheath 208 protects stent segments 202 during delivery and also prevents unintended axial displacement of the segments 202. In this embodiment, six stent segments 202 are disposed on the stent delivery catheter 200, each having a length approximately 6 mm long. Thus, in this embodiment, the delivery catheter 200 is adapted to deliver a prosthesis having a length ranging from 6 mm long, up to 36 mm long in 6 mm increments. Other lengths and quantities of stent segments may be employed and this exemplary embodiment is not meant to limit the scope of the present invention. A separator or stent valve 206 is disposed on the inner surface of outer shaft 211 and is adapted to engage prosthetic segments 202 and facilitates their deployment. Additionally, sheath 208 has an annular flange or tab 212 disposed on its outer surface that is adapted to pass through the stent valve 206 during retraction or advancement and to engage the annular flange or tab 214 disposed on the inner surface of outer shaft 211.

The length of the lesion to be treated is typically visualized by introducing contrast media into the target vessel V and observing the resulting image under a fluoroscope. Radiopaque markers 226, 228, one at the distal end of the balloon 204 and one at the distal end of the outer shaft 211 may be used to visualize the length of stent segments 202 exposed for deployment relative to the target lesion. This is accomplished by advancing the delivery catheter 200 so that radiopaque marker 226 is at the distal edge of the lesion and then outer shaft 211 is advanced until radiopaque marker 228 is at the proximal edge of the lesion. Retraction of sheath 208 engages outer shaft 211 and then both sheath 208 and outer shaft 211 are retracted distally, resulting in a number of stent segments 218 being selected to match the length of lesion L, as shown in FIG. 2B. In FIG. 2B, outer shaft 211 is advanced distally so as to select two prosthetic segments 218 having a combined length of approximately 12 mm. Additional prosthetic segments 202 may be selected if necessary, although here, four prosthetic segments 202 will remain with the delivery catheter 200. As outer shaft 211 is advanced, stent valve 206 is angled inwardly and adapted so that it will slide over sheath 208 without hindering distal motion of outer shaft 211. Advancement of outer shaft 211 may be controlled by the control mechanism 106 illustrated in FIG. 1, although other actuators may be employed. After an appropriate number of prosthetic segments have been selected for deployment, the selected segments 218 are exposed in preparation for delivery. In FIG. 2C, sheath 208 is retracted proximally and thus the selected prosthetic segments 218 are no longer constrained from expansion. As sheath 208 is retracted, its annular flange 212 slides past the stent valve 206. Retraction of sheath 208 continues until the sheath annular flange 212 engages the outer shaft annular flange 214. The two flanges 212, 214 are designed to provide a positive stop to one another. Thus, continued retraction of sheath 208 results in simultaneous retraction of outer shaft 211, as shown in FIG. 2D. Retraction of sheath 208 can be accomplished using the control mechanism 140 depicted in FIG. 1.

Additionally, once annular flange 212 on the sheath 208 has passed through stent valve 206, stent valve 206 is no longer disposed over sheath 208. The stent valve 206 now engages the distal-most prosthetic segment 202 in the group of prosthetic segments remaining with the delivery catheter 200, and this is shown in FIG. 2D. Stent valve 206 is inclined such that further retraction of sheath 208 not only retracts outer shaft 211, but stent valve 206 also retracts the group of prosthetic segments 202 that remain with the delivery catheter 200, thereby creating a spacing between prosthetic segments 202 remaining with the delivery catheter 200 and the prosthetic segments 218 to be delivered. This spacing is typically between 0.5 mm and 5 mm and is required in order to allow a balloon taper to form during balloon inflation. In addition to the inclined stent valve 206 shown in this embodiment, other stent valves are contemplated, including those disclosed in co-pending application Ser. No. ______, filed ______ (Attorney Docket No. 021629-004000US), entitled “Custom Length Stent Apparatus,” the contents of which have previously been incorporated herein by reference.

Referring now to FIG. 2E, the selected prosthetic segments 218 are deployed. Expandable member 204, here, a balloon, is inflated with a fluid such as contrast media and/or saline to achieve an expanded diameter 222. Radial expansion of the balloon 204 to an enlarged diameter 222 correspondingly expands stent segments 218 to an expanded diameter 220 outward against the vessel wall across lesion L. The proximal portion of expandable member 204 is constrained by sheath 208 and radiopaque marker 228, preventing its expansion and deployment of the remaining prosthetic segments 202. After stent segments 220 are deployed, expanded balloon 222 is deflated and removed from the deployed stent segments 220, leaving them in a plastically deformed, expanded configuration in the vessel V, at the site of the lesion, L. This is shown in FIG. 2F. Stent segments 202 remain with the delivery catheter 200 which is then removed and retracted from the vessel V.

While the exemplary embodiments have been described in some detail for clarity of understanding and by way of example, a variety of additional modifications, adaptations and changes may be clear to those of skill in the art. Hence, the scope of the present invention is limited solely by the appended claims.

What is claimed is:

1. An apparatus for delivering prosthetic segments in a body lumen, the apparatus comprising:
an elongated flexible member having a proximal end and a distal end;
a plurality of prosthetic segments releasably arranged axially along the elongated flexible member near the distal end;
a sheath slidably disposed over at least a portion of the prosthetic segments;
an outer shaft slidably disposed over at least a portion of the sheath; and
a separator disposed on the outer shaft and biased into engagement with at least one prosthetic segment so that the outer shaft may be retracted to separate a proximal group of prosthetic segments from a distal group of the prosthetic segments which are to be deployed in the body lumen, wherein the sheath is positionable between the separator and the prosthetic segments to selectively disengage the separator from the prosthetic segments.

2. An apparatus as in claim 1, wherein the separator exerts substantially greater axial force against the prosthetic segments when the separator is retracted proximally than when the separator is advanced distally.

3. An apparatus as in claim 1, wherein the sheath is configured to engage the outer shaft such that retraction of the sheath retracts the outer shaft.

4. An apparatus as in claim 2, wherein the sheath comprises an annular flange.

5. An apparatus as in claim 2, wherein the outer shaft comprises an annular flange.

6. An apparatus as in claim 1, further comprising an expandable member near the distal end of the elongated flexible member.

7. An apparatus as in claim 6, wherein the expandable member is a balloon.

8. An apparatus as in claim 1, wherein the prosthetic segments are balloon expandable.

9. An apparatus as in claim 1, wherein the prosthetic segments are self-expanding.

10. An apparatus as in claim 1, wherein the plurality of prosthetic segments carry a therapeutic agent adapted to being released therefrom.

11. An apparatus as in claim 10, wherein the therapeutic agent comprises an anti-restenosis agent.

12. An apparatus as in claim 1, wherein the plurality of prosthetic segments have a length in the range from about 2 mm to about 10 mm.

13. An apparatus as in claim 1, wherein the plurality of prosthetic segments have a length about 3 mm to 6 mm.

14. An apparatus as in claim 1, wherein each of the plurality of prosthetic segments have two or more lengths.

15. An apparatus as in claim 1, wherein each of the plurality of prosthetic segments have substantially the same length.

16. An apparatus as in claim 1, wherein the plurality of prosthetic segments have interleaved ends prior to deployment.

17. An apparatus as in claim 1, wherein the plurality of prosthetic segments are spaced apart prior to deployment to allow the separator to engage the prosthetic segments at their distal ends.

18. An apparatus as in claim 1, wherein the separator comprises a plurality of resilient fingers projecting radially inward.

19. An apparatus as in claim 18, wherein at least some of the fingers are inclined so that free ends of the fingers point proximally allowing the fingers to pass over the prosthetic segments as the separator is advanced distally, but to engage a prosthetic segment when the separator is retracted proximally.

20. An apparatus as in claim 18, wherein at least some of the fingers are composed of metal.

21. An apparatus as in claim 18, wherein at least some of the fingers are composed of a polymer.

22. An apparatus as in claim 19, wherein at least some of the fingers comprise a radiused end substantially matching the curvature of the surface of the prosthetic segments thereby providing greater contact surface so as to facilitate engagement between the prosthetic segments and the separator as the separator is retracted proximally, while allowing the separator to pass over the prosthetic segments during distal advancement of the separator.

23. An apparatus as in claim 19, wherein the separator further comprises a hinge coupled to the fingers to allow the fingers to deflect radially to facilitate passage of the separator over the sheath when the separator is advanced distally.

24. An apparatus as in claim 1, wherein the separator comprises an annular flange.

25. An apparatus as in claim 24, wherein the annular flange is tapered.

26. An apparatus as in claim 1, wherein the separator comprises a tapered conical nose.

27. An apparatus as in claim 1, wherein the separator comprises a plurality of inclined ramps disposed on an inner surface of the outer shaft.

28. An apparatus as in claim 27, wherein the inclined ramps are separated by about 90°.

29. An apparatus as in claim 1, wherein the separator comprises a compliant sharp edge.

30. A method for delivering prosthetic segments to a body lumen, the method comprising:
introducing a plurality of prosthetic segments releasably arranged axially along an elongated flexible member, into a body lumen having a lesion with a lesion length at a first treatment site;
distally advancing an outer shaft having a separator relative to a selected group of prosthetic segments selected for delivery, the selected group of prosthetic segments having a combined length that matches the lesion length;
positioning a sheath disposed between the separator and the prosthetic segments until the separator is allowed to engage the prosthetic segments;
retracting the outer shaft so as to create a spacing between prosthetic segments in the selected group and one or more prosthetic segments remaining with the elongated flexible member; and
deploying the selected group of prosthetic segments at the first treatment site.

31. A method as in claim 30, wherein the sheath is configured to engage the outer shaft such that retraction of the sheath retracts the outer shaft.

32. A method as in claim 30, wherein the plurality of prosthetic segments are introduced into a blood vessel.

33. A method as in claim 30, wherein the plurality of prosthetic segments are balloon expandable.

34. A method as in claim 30, wherein the plurality of prosthetic segments are self-expanding.

35. A method as in claim 30, wherein deploying the selected group of prosthetic segments comprises plastically deforming the selected group of prosthetic segments.

36. A method as in claim 35, wherein the selected group of prosthetic segments are plastically deformed with a balloon.

37. A method as in claim 30, wherein the plurality of prosthetic segments carry a therapeutic agent adapted to being released therefrom.

38. A method as in claim 37, wherein the therapeutic agent is an anti-restenosis agent.

39. A method as in claim 30, wherein the plurality of prosthetic segments have a length in the range from about 2 mm to about 10 mm.

40. A method as in as in claim 30, wherein the plurality of prosthetic segments have a length about 3 mm to 6 mm.

41. A method as in claim 30, wherein the plurality of prosthetic segments have interleaved ends prior to deployment.

42. A method as in claim 30, wherein ends of the plurality of prosthetic segments are spaced apart prior to deployment to allow the separator to engage the prosthetic segments at their distal ends.

43. A method as in claim 30, wherein the separator exerts substantially greater axial force against the prosthetic segments when the separator is retracted proximally than when the separator is advanced distally.

44. A method as in claim 30, wherein the separator comprises a plurality of resilient fingers projecting radially inward.

45. A method as in claim 44, wherein at least some of the fingers are inclined so that free ends of the fingers point proximally allowing the fingers to pass over the prosthetic segments as the separator is advanced distally but to engage a prosthetic segment when the separator is retracted proximally.

46. A method as in claim 45, wherein at least some of the fingers are composed of metal.

47. A method as in claim 45, wherein at least some of the fingers are composed of a polymer.

48. A method as in claim 45, wherein at least some of the fingers comprise a radiused end substantially matching the curvature of the surface of the prosthetic segments thereby providing greater contact surface so as to facilitate engagement between the prosthetic segments and the separator as the separator is retracted proximally while allowing the separator to pass over the prosthetic segments during distal advancement of the separator.

49. A method as in claim 45, wherein the separator further comprises a hinge coupled to the fingers, the fingers deflecting radially outward over the sheath when the separator is advanced distally.

50. A method as in claim 30, wherein the separator comprises an annular flange.

51. A method as in claim 50, wherein the annular flange is tapered.

52. A method as in claim 30, wherein the separator comprises a tapered conical nose.

53. A method as in claim 30, wherein the separator comprises a plurality of inclined ramps disposed on an inner surface of the outer shaft.

54. A method as in claim 53, wherein the inclined ramps are separated by about 90°.

55. A method as in claim 30, wherein the separator comprises a sharp compliant edge.

56. A method for selectively delivering prosthetic segments to a lesion in a treatment region of a body lumen, the method comprising:
advancing a delivery catheter through the body lumen to the treatment region, wherein a plurality of prosthetic segments are disposed axially along the delivery catheter;
distally advancing an outer shaft having a separator relative to a selected group of prosthetic segments selected for delivery, the selected group of prosthetic segments having a combined length that matches the lesion;
positioning a sheath disposed between the separator and the prosthetic segments until the separator is allowed to engage the prosthetic segments;
retracting the outer shaft so as to create a spacing between prosthetic segments in the selected group and one or more prosthetic segments remaining with the elongate flexible member; and
inflating a balloon disposed on the delivery catheter so as to deploy the group of prosthetic segments while any remaining prosthetic segments stay with the delivery catheter.

57. A method as in claim 56, wherein the sheath is configured to engage the outer shaft such that retraction of the sheath retracts the outer shaft.

58. A method as in claim 56, wherein the body lumen is a blood vessel.

59. A method as in claim 56, wherein the plurality of prosthetic segments are self-expanding.

60. A method as in claim 56, wherein the plurality of prosthetic segments carry a therapeutic agent adapted to being released therefrom.

61. A method as in claim 60, wherein the therapeutic agent is an anti-restenosis agent.

62. A method as in claim 56, wherein the plurality of prosthetic segments have a length in the range from about 2 mm to about 10 mm.

63. A method as in claim 56, wherein the plurality of prosthetic segments have a length about 3 mm to 6 mm.

64. A method as in claim 56, wherein each of the plurality of prosthetic segments have two or more lengths.

65. A method as in claim 56, wherein each of the plurality of prosthetic segments have substantially the same length.

66. A method as in claim 56, wherein the plurality of prosthetic segments have interleaved ends prior to deployment.

67. A method as in claim 56, wherein ends of the plurality of prosthetic segments are spaced apart prior to deployment to allow the separator to engage the prosthetic segments at their distal ends.

68. A method as in claim 56, wherein the separator exerts substantially greater axial force against the prosthetic segments when the separator is retracted proximally than when the separator is advanced distally.

69. A method as in claim 56, wherein the separator comprises a plurality of resilient fingers projecting radially inward.

70. A method as in claim 69, wherein at least some of the fingers are inclined so that free ends of the fingers point proximally allowing the fingers to pass over the prosthetic segments as the separator is advanced distally but will engage a prosthetic segment when the separator is retracted proximally.

71. A method as in claim 70, wherein at least some of the fingers are composed of metal.

72. A method as in claim 70, wherein at least some of the fingers are composed of a polymer.

73. A method as in claim 70, wherein at least some of the fingers comprise a radiused end substantially matching the curvature of the surface of the prosthetic segments thereby providing greater contact surface so as to facilitate engagement between the prosthetic segments and the separator as the separator is retracted proximally while allowing the separator to pass over the prosthetic segments during distal advancement of the separator.

74. A method as in claim 70, wherein the separator further comprises a hinge coupled to the fingers, the fingers deflecting radially outward over the prosthetic segments when the separator is advanced distally.

75. A method as in claim 56, wherein the separator comprises an annular flange.

76. A method as in claim 75, wherein the annular flange is tapered.

77. A method as in claim 56, wherein the separator comprises a tapered conical nose.

78. A method as in claim 56, wherein the separator comprises a plurality of inclined ramps disposed on an inner surface of an outer shaft.

79. A method as in claim 78, wherein the inclined ramps are separated by about 90°.

80. A method as in claim 56, wherein the separator comprises a sharp compliant edge.
